(19) United States
(12) Patent Application Publication
(10) Pub. No.: US 2022/0265614 A1
(43) Pub. Date: Aug. 25, 2022

(54) TREATMENT COMPRISING FXR AGONISTS

(71) Applicant: Novartis AG, Basel (CH)

(72) Inventors: Dominique BREES, Arlesheim (CH); Patricia LOPEZ, Basel (CH)

(21) Appl. No.: 17/628,838

(22) PCT Filed: Jul. 21, 2020
(86) PCT No.: PCT/IB2020/056834

§ 371 Date: Jan. 20, 2022

Related U.S. Application Data
(60) Provisional application No. 62/877,448, filed on Jul. 23, 2019.
(60) Provisional application No. 62/901,568, filed on Sep. 17, 2019.

(51) Int. Cl. A61K 31/427; A61K 45/06; A61P 1/16

(52) U.S. Cl. CPC A61K 31/427; A61K 45/06; A61P 1/16

(57) ABSTRACT

The invention provides methods of treating, preventing, or ameliorating conditions mediated by farnesoid X receptors (FXR), in particular liver diseases or intestinal diseases, e.g. NASH, comprising administering to a subject in need thereof a therapeutically effective amount of a FXR agonist.

### FIELD OF THE INVENTION

The present invention relates to methods of treating, preventing, or ameliorating conditions mediated by farnesoid X receptors (FXRs), in particular liver diseases or intestinal disease, comprising administering to a subject in need thereof a therapeutically effective amount of a FXR agonist. Furthermore, the invention is directed to the use of a farnesoid X receptor agonist (FXR agonist), such as tropifexor, for treating or preventing fibrotic or cirrhotic diseases or disorders, e.g. liver diseases or disorders.

### BACKGROUND OF THE INVENTION

Nonalcoholic fatty liver disease (NAFLD) is the most common cause of chronic liver disease in the Western world. The main stages of NAFLD are 1-simple fatty liver (steatosis); 2-non-alcoholic steatohepatitis (NASH), a more serious form of NAFLD with fat accumulation accompanied by inflammation and cell injury; 3-fibrosis, where there is a persistent inflammation in the liver resulting in the generation of fibrous scar tissue around the liver cells and blood vessels; and 4-cirrhosis; this damage is permanent and can lead to liver failure and liver cancer (hepatocellular carcinoma).

Liver transplantation is the only treatment for advanced cirrhosis with liver failure. Estimates of the worldwide prevalence of NAFLD range from 6.3% to 33% with a median of 20% in the general population. The estimated prevalence of NASH is lower, ranging from 3 to 5% (Younossi et al., Hepatology, Vol. 64, No. 1, 2016). NASH is a worldwide problem with growing prevalence over the last few decades. Over the last decade NASH has risen from uncommon to the second indication for liver transplantation in the US. It is expected to be the leading cause of transplant by 2020. NASH is highly associated with the metabolic syndrome and Type 2 diabetes mellitus. Furthermore, cardiovascular mortality is an important cause of death in NASH patients.

Development of NASH, involves several mechanisms: accumulation of fat in the liver (steatosis), inflammation of the liver, hepatocyte ballooning, and fibrosis. The NAFLD Activity Score (NAS) was developed as a tool to measure changes in NAFLD during therapeutic trials. The score is calculated as the unweighted sum of the scores for steatosis (0-3), lobular inflammation (0-3), and ballooning (0-2).

When tested in NASH patients, obeticholic acid (OCA), a bile acid mimetic, showed efficacy, in particular a significant improvement in NAS, i.e. strong impact on steatosis with additional effects on lobular inflammation and ballooning. But OCA long term administration raises safety concerns because it was associated with pruritus, as well as with lipid abnormalities, i.e. increased low density lipoprotein (LDL) cholesterol (see Results from REGENERATE (NCT02548351), A Phase 3 International, Randomized, Placebo-Controlled Study Evaluating Obeticholic Acid Treatment for NASH, EASL 2019 Apr. 10-14 Vienna). Pruritus is the most common adverse effect in the patients treated with OCA. This side effect reported in association with the treatment with the FXR agonist OCA may be requiring dose adjustment and/or discontinuation of the administration. Pruritus may also be managed in most patients by i.e. use of bile acid sequestrants, antihistamines, dose reduction, or symptomatic treatment. Furthermore, to avoid the risk of adverse cardiovascular events, concomitant administration of statins may be required for long-term treatment of NASH patients treated with OCA.

The FXR agonist tropifexor (Tully et al, J Med Chem 2017;60:9960-9973) is currently tested in nonalcoholic steatohepatitis patients with fibrosis (see NCT02855164 study). The compound was disclosed for the first time in WO 2012/087519 (Example 1, compound 1-IB of the table on page 125) and it is known under the name LJN452.

Currently there is no approved therapy for NASH. Therefore, there is a need to provide treatments for fibrotic/cirrhotic diseases or disorders, e.g. liver diseases or disorders, e.g. NASH, which can address the different aspects of these complex conditions, while demonstrating an acceptable safety and/or tolerability profile.

### SUMMARY OF THE INVENTION

The invention relates to methods of treating, preventing, or ameliorating conditions mediated by farnesoid X receptors (FXR), in particular liver diseases or intestinal diseases, e.g. NASH, comprising administering to a subject in need thereof a therapeutically effective amount of a FXR agonist, wherein the administration of the FXR agonist to said subject is occurring in the evening.

The invention relates to methods of treating, preventing, or ameliorating conditions mediated by FXRs, in particular liver diseases or intestinal diseases, e.g. NASH, comprising administering to a subject in need thereof a therapeutically effective amount of a FXR agonist of formula

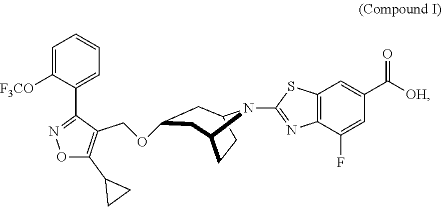

i.e. 2-[(1R,3r,5S)-3-({5-cyclopropyl-3-[2-(trifluoromethoxy)phenyl]-1,2-oxazol-4-yl}methoxy)-8-azabicyclo[3.2.1]octan-8-yl]-4-fluoro-1,3-benzothiazole-6-carboxylic acid), in free form, or a pharmaceutically acceptable salt thereof or an amino acid conjugate thereof, also known under its INN tropifexor, wherein the administration of the FXR agonist to said subject is occurring in the evening.

The invention provides new treatment regimens containing at least one FXR agonist, such as for example tropifexor, wherein the administration of the FXR agonist is occurring in the evening. The treatment regimens according to the present invention offer the benefit of a high therapeutic efficacy while having low incidence of side effects, such as itching and/or lipid abnormalities (e.g. increased LDL cholesterol), which are, observed while using conventional treatment regimen. These treatment regimens further provide subjects with a convenient once daily dosing, thus supporting patient compliance.

### BRIEF DESCRIPTION OF DRAWINGS

FIG. 1 provides the study design of a 2 week study in Cynomolgus monkey treated with the FXR agonist LJP305 (compound described in Tully et al, J Med Chem 2017;60:9960-9973).

FIG. 2 shows the 7α-hydroxy-4-cholesten-3-one (C4) measurements in the different groups of the 2 week study in Cynomolgus monkey treated with the FXR agonist LJP305.

FIG. 3 shows the cholic acid (CA) measurements in the different groups of the 2 week study in Cynomolgus monkey treated with the FXR agonist LJP305.

FIG. 4 shows the levels of chenodeoxycholic acid (CDCA) in the different groups of the 2 week study in Cynomolgus monkey treated with the FXR agonist LJP305.

FIG. 5 shows that in vitro human hepatocytes treated with the FXR agonists OCA and cilofexor have decreased LDL uptake.

### DETAILED DESCRIPTION OF THE INVENTION

It has been found that administering a FXR agonist to a subject in need thereof in the evening, for example shortly before or at bedtime, is beneficial for therapeutic efficacy and for safety (such as reducing itch and/or lipid abnormalities).

7α-hydroxy-4-cholesten-3-one (C4) is an intermediate bile acid precursor directly produced by cholesterol 7-alpha-monooxygenase or cytochrome P450 7A1 (Cyp7A1). C4 has 2 peaks in the plasma, one around 1 pm and the other around 9 pm (Galman et al, Gastroenterology 2005; 129:1445-1453). These peaks are corresponding to timing of the larger meals of the day; the bile acids being needed for digestion. This implies that Cyp7A1, which produces C4, as well as FXR which is the counter mechanism for the production, are following the same daily rhythms in human. Administration of an FXR agonist in the evening (e.g. from about 6 pm to about 12 pm, preferably from about 8 pm to about 11 pm, preferably around 9 pm), should allow the FXR agonist to stimulate the system when the activity of the transcription factor FXR is decreasing hence allowing for a more prolonged effect of FXR agonist during the night when normally FXR activity is at the lowest. Such a dosing schedule should increase the efficacy of the FXR agonist.

Chenodeoxycholic acid (CDCA), major primary bile acid, is primarily responsible for the bile acid induced itch (Alemi et al, The Journal of Clinical Investigation 2013; 123:1513-1530). It has been found that the FXR agonist-induced itch is caused by a sustained inhibition of Cyp7a1 causing a shutdown of the C4/bile acid production leading to the activation of the alternate bile acid pathway via activation of Cyp27a1, this leading to the production of prurigenic CDCA bile acid. Administration of an FXR agonist when the enzymatic activity of Cyp7A1 is at the lowest should minimize the effect of FXR-mediated inhibition of Cyp7A1 and consequent activation of the alternate bile acid pathway.

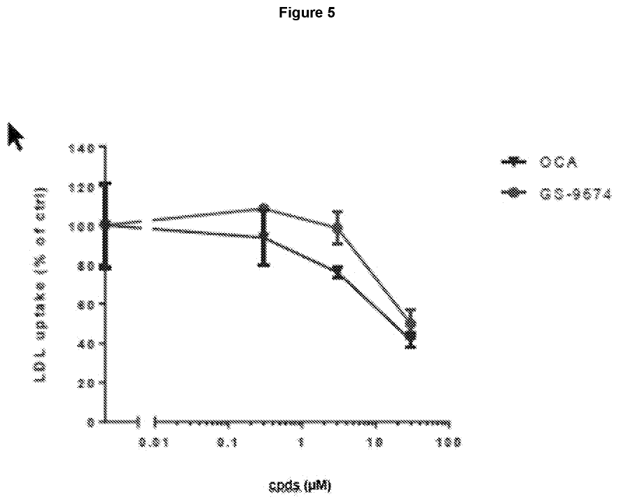

In addition, FXR agonist treatments have been associated with lipid abnormalities, including increases in peripheral LDL (Neuschwander-Tetri et al, The Lancet 2015; 385: 956-965). The reduction of the bile acid pathway by FXR agonists could lead to intracytoplasmic increase in cholesterol in the hepatocytes. Increase cholesterol in hepatocytes is associated with a counter mechanism of decrease LDL receptor on the surface of the cells (Goldstein et al Circulation. 1987 September;76(3):504-7). Such a decrease in the LDL receptor on the surface of the hepatocytes will ultimately result in an increase in circulating LDL; the phenotype observed in the clinics. We have demonstrated in vitro, using in vitro human hepatocytes, that FXR agonists reduce the LDL uptake by hepatocytes in a dose dependent manner (FIG. 5). Those data indicates that blocking the Cyp7A1 and the bile acid pathway leads to the peripheral increase in LDL. To mitigate the increase in circulating LDL, it is proposed to administer a FXR agonist to the subjects in need thereof in the evening (e.g. from about 6 pm to about 12 pm, preferably from about 8 pm to about 11 pm, preferably around 9 pm) to reduce the impact of the FXR agonist on circulating LDL.

Various (enumerated) embodiments of the present invention are described herein. It will be recognized that features specified in each embodiment may be combined with other specified features to provide further embodiments of the present disclosure.

### Embodiments (a)

1a: A FXR agonist for use in the treatment of a condition mediated by Farnesoid X receptor (FXR), in particular a liver disease or an intestinal disease, wherein the FXR agonist is administered once daily at a therapeutically effective dose, and wherein the FXR agonist is administered in the evening.

2a: A FXR agonist for use in the prevention of a condition mediated by Farnesoid X receptor (FXR), in particular a liver disease or an intestinal disease, wherein the FXR agonist is administered once daily at a therapeutically effective dose, and wherein the FXR agonist is administered in the evening.

3a. A FXR agonist for use in the treatment, stabilization or lessening the severity or progression of a non-alcoholic fatty liver disease (NAFLD), e.g. NASH, in a subject in need thereof, wherein the FXR agonist is administered once daily at a therapeutically effective dose, and wherein the FXR agonist is administered in the evening.

4a. A FXR agonist for use in the treatment, stabilization or lessening the severity or progression of an intestinal disease in a subject in need thereof, wherein the FXR agonist is administered once daily at a therapeutically effective dose, and wherein the FXR agonist is administered in the evening.

5a. A FXR agonist for use in the slowing, arresting, or reducing the development of a chronic liver disease or disorder, e.g. NAFLD, NASH, liver fibrosis or PBC, in a subject in need thereof, wherein the FXR agonist is administered once daily at a therapeutically effective dose, and wherein the FXR agonist is administered in the evening.

6a. The FXR agonist for use according to any of Embodiments 1a to Embodiment 5a, wherein the FXR agonist is selected from tropifexor, obeticholic acid, nidufexor, cilofexor, TERN-101, EDP-305, PXL007, AGN242266 and MET409.

7a. Tropifexor, e.g. in free form, or a salt thereof, or an amino acid conjugate thereof, for use in the treatment or prevention of a condition mediated by FXR; in particular a liver disease or an intestinal disease, wherein tropifexor is administered once daily, at a therapeutically effective dose, and wherein tropifexor is administered in the evening.

8a. Tropifexor, e.g. in free form, or a salt thereof, or an amino acid conjugate thereof, for use in the treatment or prevention of a condition mediated by FXR; in particular a liver disease or an intestinal disease, wherein tropifexor is to be administered once daily, in the evening, at a dose of about 90 μg to about 250 μg, e.g. of about 140 μg to about 200 μg.

9a. Tropifexor, e.g. in free form, or a salt thereof, or an amino acid conjugate thereof, for use in the treatment or prevention of non-alcoholic fatty liver disease (NAFLD), non-alcoholic steatohepatitis (NASH), liver fibrosis or PBC, wherein tropifexor is administered once daily, at a therapeutically effective dose, and wherein tropifexor is administered in the evening.

10a. Tropifexor, e.g. in free form, or a salt thereof, or an amino acid conjugate thereof, for use in the treatment or prevention of non-alcoholic fatty liver disease (NAFLD), or of non-alcoholic steatohepatitis (NASH), wherein tropifexor is to be administered once daily, at a dose of about 90 μg to about 250 μg, or of about 140 μg to about 200 μg, and wherein tropifexor is administered in the evening.

11a. Tropifexor for use according to any of Embodiments 7a to 10a, wherein tropifexor is to be administered at a daily dose of about 140 μg.

12a. The FXR agonist for use according to any one of Embodiments 1a to 11a, wherein said evening administration ameliorates the efficacy associated with the administration of the FXR agonist.

13a. The FXR agonist for use according to any one of Embodiments 1a to 12a, wherein said evening administration reduces the risk of side effects, e.g. pruritus, associated with the administration of the FXR agonist.

14a. The FXR agonist for use according to any one of Embodiments 1a to 11a, wherein said evening administration reduces the risk of side effects, e.g. lipid abnormality, associated with administration of the FXR agonist.

15a. The FXR agonist for use according to any one of Embodiments 1a to 14a, wherein said administration comprises resolution of steatohepatitis.

16a The FXR agonist for use according to any one of Embodiments 1a to 14a, wherein said administration comprises improvement in liver fibrosis.

17a The FXR agonist for use according to any one of Embodiments 1a to 14a, wherein said administration comprises resolution of steatohepatitis and improvement in liver fibrosis.

### Embodiments (b)

1b. A method for the treatment of a condition mediated by Farnesoid X receptor (FXR), in particular a liver disease or an intestinal disease, in a subject in need thereof, comprising administering once daily to said subject a therapeutically effective amount of a FXR agonist, wherein the FXR agonist is administered in the evening.

2b. A method for the prevention of a condition mediated by Farnesoid X receptor (FXR), in particular a liver disease or an intestinal disease, in a subject in need thereof, comprising administering once daily to said subject a therapeutically effective amount of a FXR agonist, wherein the FXR agonist is administered in the evening.

3b. A method for the treatment, stabilization or lessening the severity or progression of a non-alcoholic fatty liver disease (NAFLD) in a subject in need thereof, comprising administering once daily to said subject a therapeutically effective amount of a FXR agonist, wherein the FXR agonist is administered in the evening.

4b. A method for the treatment, stabilization or lessening the severity or progression of an intestinal disease in a subject in need thereof, comprising administering once daily to said subject a therapeutically effective amount of a FXR agonist, wherein the FXR agonist is administered in the evening.

5b. A method for the treatment, stabilization or lessening the severity or progression of a non-alcoholic steatohepatitis (NASH) in a subject in need thereof comprising administering once daily to said subject a therapeutically effective amount of an FXR agonist, wherein the FXR agonist is administered in the evening.

6b. A method for slowing, arresting, or reducing the development of a chronic liver disease or disorder, e.g. NAFLD, NASH, liver fibrosis or PBC, in a subject in need thereof, comprising administering once daily to said subject a therapeutically effective amount of a FXR agonist, wherein the FXR agonist is administered in the evening.

7b. A method for reducing cirrhosis or fibrosis in a subject having a disease that is non-alcoholic fatty liver disease (NAFLD) or non-alcoholic steatohepatitis (NASH), comprising administering once daily to said subject a therapeutically effective amount of a FXR agonist, wherein the FXR agonist is administered in the evening.

8b. The method according to any one of Embodiments 1b to 7b, wherein said method further comprises lack of worsening of the subject's NAFLD as defined by Activity (NAS) score, lack of worsening of the subject's Steatosis, Activity and Fibrosis (SAF) Activity score, reduction of liver fat in said subject, improvement in subject's Steatosis, improvement in subject's ballooning, NAFLD resolution, NAFLD resolution without worsening of fibrosis, reduction of fibrosis without NAFLD worsening, reduction of ALT levels in said subject, reduction of AST levels in said subject, reduction of HbA1c levels in said subject, lack of subject's progression to Cirrhosis, inhibiting progression of Non-Alcoholic Fatty Liver Disease (NAFLD) and/or Non-Alcoholic Steatohepatitis (NASH), or any combination thereof.

9b. The method according to any one of Embodiments 1b to 8b, wherein the FXR agonist is selected from tropifexor, obeticholic acid, nidufexor, cilofexor, TERN-101, EDP-305, PXL007, AGN242266 and MET409.

10b. The method according to Embodiment 9b, wherein the FXR agonist is obeticholic acid.

11b. The method according to Embodiment 10b, wherein obeticholic acid is administered at a daily dose of about 5 mg, of about 10 mg, of about 15 mg, of about 20 mg, of about 25 mg, of about 30 mg, of about 40 mg or of about 50 mg.

12b. The method according to Embodiment 9b, wherein the FXR agonist is tropifexor.

13b. The method according to Embodiment 12b, wherein tropifexor is administered at a daily dose of about 90 μg to about 250 μg, e.g. of about 140 μg to about 200 μg.

14b. The method according to Embodiment 12b, wherein tropifexor is to be administered at a dose of about 90 μg/day, of about 140 μg/day, of about 150 μg/day, of about 160 μg/day, of about 170 μg/day, of about 180 μg/day, of about 190 μg/day, of about 200 μg/day, of about 210 μg/day, of about 220 μg/day, of about 230 μg/day, of about 240 μg/day or of about 250 μg/day.

15b. The method according to Embodiment 12b wherein tropifexor is to be administered at a daily dose of about 140 μg.

16b. The method according to any one of Embodiments 1b to 15b, wherein said evening administration ameliorates the efficacy associated with administration of the FXR agonist.

17b. The method according to any one of Embodiments 1b to 16b, wherein said evening administration reduces the risk of side effects, e.g. pruritus, associated with administration of the FXR agonist.

18b. The method according to any one of Embodiments 1b to 16b, wherein said evening administration reduces the risk of side effects, e.g. lipid abnormality, associated with administration of the FXR agonist.

19b. The method according to any one of Embodiments 1b to 15b, wherein said administration comprises resolution of steatohepatitis, e.g. NASH.

20b. The method according to any one of Embodiments 1b to 15b, wherein said administration comprises improvement in liver fibrosis.

21b. The method according to any one of Embodiments 1b to 15b, wherein said administration comprises resolution of steatohepatitis, e.g. NASH, and improvement in liver fibrosis.

### Embodiments (c)

1c. A pharmaceutical composition comprising a FXR agonist, or a pharmaceutically acceptable salt thereof, and at least one pharmaceutically acceptable excipient, for use in the treatment of a condition mediated by Farnesoid X receptor (FXR), in particular liver disease or intestinal disease, in a subject in need thereof, comprising a therapeutically effective amount of at least one FXR agonist, wherein the pharmaceutical composition is to be administered once daily, in the evening.

2c. A pharmaceutical composition comprising an FXR agonist for use according to any of Embodiments 1a to 17a, and at least one pharmaceutically acceptable excipient.

### Embodiments (d)

1d. Use of FXR agonist as defined in any one of Embodiments 1a to 17a, or a pharmaceutically acceptable salt thereof, for the manufacture of a medicament for the treatment of a condition mediated by Farnesoid X receptor (FXR), in particular a liver disease or an intestinal disease.

2d. Use of tropifexor in the manufacture of a medicament for treating or preventing a condition mediated by Farnesoid X receptor (FXR), wherein tropifexor is to be administered once daily, at a dose daily dose, of about 90 μg to about 250 μg, about 140 μg to about 200 μg, and wherein tropifexor is administered in the evening.

3d. The use of tropifexor according to Embodiment 2d, wherein said condition mediated by FXR is non-alcoholic fatty liver disease (NAFLD), non-alcoholic steatohepatitis (NASH), cholelithiasis or liver fibrosis.

4d. The use of tropifexor according to Embodiment 3d, wherein the condition mediated by FXR is NASH.

### Embodiments (e)

1 e. Use of a pharmaceutical composition comprising an FXR agonist according to any one of Embodiment 1a to 17a, or a pharmaceutically acceptable salt thereof, and at least one pharmaceutically acceptable excipient, for the manufacture of a medicament for the treatment of a condition mediated by Farnesoid X receptor (FXR), in particular liver disease or intestinal disease.

A FXR agonist, a method, a pharmaceutical composition, or a use, according to any one of above listed Embodiments, for treating or preventing non-alcoholic steatohepatitis (NASH), and wherein NASH is mild to moderate with fibrosis level F2-F3.

A FXR agonist, a method, a pharmaceutical composition, or a use, according to any one of above listed Embodiments, wherein NASH is confirmed based on liver biopsy (also called biopsy-proven NASH) and NASH is mild to moderate with fibrosis level F2-F3.

A FXR agonist, or a method according, a pharmaceutical composition, or a use, according to any one of the above listed Embodiments, wherein presence of NASH has been demonstrated by:
i) Histologic evidence of NASH based on liver biopsy obtained 2 years or less before treatment with a FXR agonist according to any one of the above Embodiments, with a diagnosis consistent with NASH, fibrosis level F1, F2, F3 or F4, no diagnosis of alternative chronic liver diseases, or
ii) Phenotypic diagnosis of NASH, or
iii) Noninvasive, disease-specific biomarkers.

Tropifexor is administered at a dose (e.g. daily dose) of about 90 μg to about 250 μg, e.g. of about 140 μg to about 200 μg. Obeticholic acid is administered at a daily dose of about 5 mg, of about 10 mg, of about 15 mg, of about 20 mg, of about 25 mg, of about 30 mg, of about 40 mg or of about 50 mg.

In some aspects, the FXR agonists as defined herein, are provided for the treatment of a disease or disorder mediated by FXR, e.g. a liver disease or disorder, e.g. a chronic liver disease or disorder, e.g. a disease or disorder selected from the group consisting of cholestasis, intrahepatic cholestasis, estrogen-induced cholestasis, drug-induced cholestasis, cholestasis of pregnancy, parenteral nutrition-associated cholestasis, primary biliary cirrhosis (PBC), primary sclerosing cholangitis (PSC), progressive familiar cholestasis (PFIC), non-alcoholic fatty liver disease (NAFLD), non-alcoholic steatohepatitis (NASH), drug-induced bile duct injury, gallstones, liver cirrhosis, alcohol-induced cirrhosis, cystic fibrosis-associated liver disease (CFLD), bile duct obstruction, cholelithiasis, liver fibrosis, renal fibrosis, dyslipidemia, atherosclerosis, diabetes, diabetic nephropathy, colitis, newborn jaundice, prevention of kernicterus, veno-occlusive disease, portal hypertension, metabolic syndrome, hypercholesterolemia, intestinal bacterial overgrowth, erectile dysfunction, progressive fibrosis of the liver caused by any of the diseases above or by infectious hepatitis, e.g. NAFLD, NASH, hepatic fibrosis, hepatosteatis or PBC.

In yet another aspect, a pharmaceutical unit dosage form composition comprising about 90 μg, about 140 μg, about 150 μg, about 160 μg, about 170 μg, about 180 μg, about 190 μg, about 200 μg, about 210 μg, about 220 μg, about 230 μg, about 240 μg or about 250 μg of tropifexor suitable for oral administration once daily, in the evening, or shortly before or at bedtime. Such unit dosage form compositions may be in a form selected from a liquid, a tablet, a capsule. Also these unit dosage form compositions are for use in treating a chronic liver disease, e.g. non-alcoholic fatty liver disease (NAFLD), non-alcoholic steatohepatitis (NASH), drug-induced bile duct injury, gallstones, liver cirrhosis, alcohol-induced cirrhosis, cystic fibrosis, bile duct obstruction, cholelithiasis, liver fibrosis, e.g. for use in treating non-alcoholic steatohepatitis (NASH), e.g. for use in treating phenotypic non-alcoholic steatohepatitis (NASH).

In yet another aspect, the FXR agonists as defined herein are provided for preventing or delaying progression of a chronic liver disease or disorder to a more advanced stage or a more serious condition thereof, e.g. for preventing or delaying progression of a chronic liver disease or disorder selected from the group consisting of NAFLD, NASH, hepatic fibrosis and PBC.

### Definitions

For purposes of interpreting this specification, the following definitions will apply and whenever appropriate, terms used in the singular will also include the plural and vice versa.

As used herein, the term “about” in relation to a numerical value x means +/−10%, unless the context dictates otherwise.

As used herein, a “FXR agonist”/“FXR agonists” refer to any agent that is capable of binding and activating farnesoid X receptor (FXR) which may be referred to as bile acid receptor (BAR) or NR1H4 (nuclear receptor subfamily 1, group H, member 4) receptor. FXR agonist may act as agonists or partial agonists of FXR. The agent may be e.g. a small molecule, an antibody or a protein, preferably a small molecule. The activity of a FXR agonist may be measured by several different methods, e.g. in an in vitro assay using the fluorescence resonance energy transfer (FRET) cell free assay as described in Pellicciari, et al. Journal of Medicinal Chemistry, 2002 vol. 15, No. 45:3569-72.

The FXR agonist as used herein refers, for example, to compounds disclosed in: WO2016/096116, WO2016/127924, WO2017/218337, WO2018/024224, WO2018/075207, WO2018/133730, WO2018/190643, WO2018/214959, WO2016/096115, WO2017/118294, WO2017/218397, WO2018/059314, WO2018/085148, WO2019/007418, CN109053751, CN104513213, WO2017/128896, WO2017/189652, WO2017/189663, WO2017/189651, WO2017/201150, WO2017/201152, WO2017/201155, WO2018/067704, WO2018/081285, WO2018/039384, WO2015/138986, WO2017/078928, WO2016/081918, WO2016/103037, WO2017/143134.

The FXR agonist is preferably selected from: tropifexor, nidufexor, obeticholic acid (6α-ethyl-chenodeoxycholic acid), cilofexor (GS-9674, Px-102),

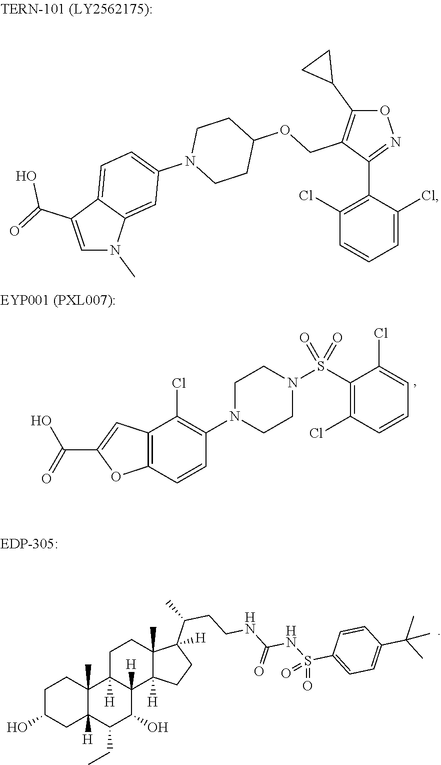

As used herein, the terms “salt” or “salts” refer to an acid addition or base addition salt of a compound of the invention. “Salts” include in particular “pharmaceutical acceptable salts”, and both can be used interchangeably herein.

As used herein, the term “pharmaceutically acceptable” means a nontoxic material that does not substantially interfere with the effectiveness of the biological activity of the active ingredient(s).

As used herein the term “prodrug” refers to a compound that is converted in vivo to the compounds of the present invention. A prodrug is active or inactive. It is modified chemically through in vivo physiological action, such as hydrolysis, metabolism and the like, into a compound of this invention following administration of the prodrug to a subject. The suitability and techniques involved in making and using pro-drugs are well known by those skilled in the art. Suitable prodrugs are often pharmaceutically acceptable ester derivatives.

As used herein, the terms “subject” or “subjects” refer to a mammalian organism, preferably a human being, who is diseased with the condition (i.e. disease or disorder) of interest and who would benefit from the treatment, e.g. a patient.

As used herein, a subject is “in need of” a treatment if such subject would benefit biologically, medically or in quality of life from such treatment.

As used herein, the term “treat”, “treating” or “treatment” of any disease or disorder refers in one embodiment to ameliorating the disease or disorder (i.e. slowing or arresting or reducing the development of the disease or at least one of the clinical symptoms or pathological features thereof). In another embodiment “treat”, “treating” or “treatment” refers to alleviating or ameliorating at least one physical parameter or pathological features of the disease, e.g. including those, which may not be discernible by the subject. In yet another embodiment, “treat”, “treating” or “treatment” refers to modulating the disease or disorder, either physically, (e.g. stabilization of at least one discernible or non-discernible symptom), physiologically (e.g. stabilization of a physical parameter) or both. In yet another embodiment, “treat”, “treating” or “treatment” refers to preventing or delaying the onset or development or progression of the disease or disorder, or of at least one symptoms or pathological features associated thereof. In yet another embodiment, “treat”, “treating” or “treatment” refers to preventing or delaying progression of the disease to a more advanced stage or a more serious condition, such as e.g. liver cirrhosis; or to preventing or delaying a need for liver transplantation.

As used herein, the term “nonalcoholic fatty liver disease” (NAFLD) may refer to nonalcoholic fatty liver (NAFL), noncirrhotic NASH, and NASH with cirrhosis.

For example, “treating” NASH may refer to ameliorating, alleviating or modulating at least one of the symptoms or pathological features associated with NASH; e.g. hepatosteatosis, hepatocellular ballooning, hepatic inflammation and fibrosis; e.g. may refer to slowing progression, reducing or stopping at least one of the symptoms or pathological features associated with NASH, e.g. hepatosteatosis, hepatocellular ballooning, hepatic inflammation and fibrosis. It may also refer to preventing or delaying liver cirrhosis or a need for liver transplantation, e.g. slow the progress of, halt, or reverse disease progression and improve clinical outcomes (i.e., prevent progression to cirrhosis and 283 cirrhosis complications, reduce the need for liver transplantation, and improve survival)

Also “treating” NASH may refer to slow the progress of, halt, or reverse disease progression and improve clinical outcomes i.e., prevent progression to cirrhosis and Resolution of steatohepatitis and no worsening of liver fibrosis on NASH clinical research network (CRN) histological score.

The treatment of NASH includes:
“Resolution of steatohepatitis” is defined as absence of fatty liver disease or isolated or simple steatosis without steatohepatitis and a NAS score of 0-1 for inflammation, 0 for ballooning, and any value for steatosis; cirrhosis complications, reduction in the need for liver transplantation, and improved survival;
Or Improvement in liver fibrosis greater than or equal to one stage (NASH CRN histological score) and no worsening of steatohepatitis (e.g. defined as no increase in NAS for ballooning, inflammation, or steatosis);
Or Both resolution of steatohepatitis and improvement in fibrosis (as defined above). “Treating” or “treatment” of NAFLD or NASH in a human includes one or more of:

a) Reducing the risk of developing NAFLD or NASH, i.e., causing clinical symptoms of NAFLD or NASH not to develop in a subject who may be predisposed to NAFLD or NASH

b) Inhibiting NAFLD or NASH, i.e., arresting or reducing the development of NALFD or NASH or its clinical symptoms; and

c) Relieving NAFLD or NASH, i.e., causing regression, reversal, or amelioration of the NAFLD or NASH or reducing number, frequency, duration or severity of its clinical symptoms.

As used herein, the term “prevent”, “preventing” or “prevention” in connection to a disease or disorder refers to the prophylactic treatment of a subject who is at risk of developing a condition (e.g., specific disease or disorder or clinical symptom thereof) resulting in a decrease in the probability that the subject will develop the condition.

As used herein, the term “therapeutically effective amount” refers to an amount of the compound, which is sufficient to achieve the stated effect. Accordingly, a therapeutically effective amount used for the treatment or prevention of a liver disease or disorder as hereinabove defined is an amount sufficient for the treatment or prevention of such a disease or disorder.

By “therapeutic regimen” is meant the pattern of treatment of an illness, e.g., the pattern of dosing used during the treatment of the disease or disorder.

As used herein, the term “liver disease or disorder” encompasses one, a plurality, or all of non-alcoholic fatty liver disease (NAFLD), non-alcoholic steatohepatitis (NASH), drug-induced bile duct injury, gallstones, liver cirrhosis, alcohol-induced cirrhosis, cystic fibrosis-associated liver disease (CFLD), bile duct obstruction, cholelithiasis and liver fibrosis.

As used herein, the term NAFLD may encompass the different stages of the disease: hepatosteatosis, NASH, fibrosis and cirrhosis.

As used herein, the term NASH may encompass steatosis, hepatocellular ballooning and lobular inflammation.

As herein defined, “combination” refers to either a fixed combination in one unit dosage form (e.g., capsule, tablet, or sachet), free (i.e. non-fixed) combination, or a kit of parts for the combined administration where a FXR agonist, such as tropifexor, and the one or more additional therapeutic agents may be administered independently at the same time or separately within time intervals, especially where these time intervals allow that the combination partners show a cooperative, e.g. synergistic effect.

The terms “co-administration” or “combined administration” or the like as utilized herein are meant to encompass administration of an additional therapeutic agent to a single subject in need thereof (e.g. a subject), and the additional therapeutic agent are intended to include treatment regimens in which the FXR agonist and additional therapeutic agent are not necessarily administered by the same route of administration and/or at the same time. Each of the components of the combination of the present invention may be administered simultaneously or sequentially and in any order. Co-administration comprises simultaneous, sequential, overlapping, interval, continuous administrations and any combination thereof.

The term “pharmaceutical combination” as used herein means a pharmaceutical composition that results from the combining (e.g. mixing) of more than one active ingredient and includes both fixed and free combinations of the active ingredients.

The term “fixed combination” means that the active ingredients are administered to a subject simultaneously in the form of a single entity or dosage.

The term “free combination” means that the active ingredients as hereindefined are administered to a subject as separate entities either simultaneously, concurrently or sequentially with no specific time limits, and in any order, wherein such administration provides therapeutically effective levels of the compounds in the subject's body.

By “simultaneous administration”, it is meant that the active ingredients as herein defined, are administered on the same day. The active ingredients can be administered at the same time (for fixed or free combinations), or one at a time (for free combinations).

According to the invention, “sequential administration”, may mean that during a period of two or more days of continuous co-administration only one of active ingredients as herein defined, is administered on any given day.

By “overlapping administration”, it is meant that during a period of two or more days of continuous co-administration, there is at least one day of simultaneous administration and at least one day when only one of active ingredients as herein defined, is administered.

By “continuous administration”, it is meant a period of co-administration without any void day. The continuous administration may be simultaneous, sequential, or overlapping, as described above.

As used herein, the term “qd” means a once daily administration.

The term “dose” refers to a specified amount of a drug administered at one time. As used herein, the dose is the amount of the drug that elicits a therapeutic effect. The dose would, for example, be declared on a product package or in a product information leaflet. For example, for tropifexor, the term “dose” when used in relation to tropifexor is the amount of tropifexor in free form. Since tropifexor can be present in the form of a salt or of an amino acid conjugate, the amount of the respective salt former (e.g. the respective acid) or of the amino acid, has to be added accordingly.

### Modes of administration

The pharmaceutical composition of the invention can be formulated to be compatible with its intended route of administration (e.g. oral compositions generally include an inert diluent or an edible carrier). Other non-limiting examples of routes of administration include parenteral (e.g. intravenous), intradermal, subcutaneous, oral (e.g. inhalation), transdermal (topical), transmucosal, and rectal administration. The pharmaceutical compositions compatible with each intended route are well known in the art.

### Timing of the Administration

The FXR agonist of the invention, as herein defined in above listed embodiments, is administered in the evening.

In one embodiment, the term “administration in the evening” is generally defined as administration any time from about 6 pm to about 12 pm, e.g. from about 8 pm to about 11 pm, preferably around 9 pm. Administration in the evening may be before the evening meal, with the evening meal or after the evening meal.

In one embodiment, the term “administration in the evening” refers to administration shortly before or at bedtime. In one embodiment, the term “administration in the evening” refers to administration shortly before bedtime. In one embodiment, the term “administration in the evening” refers to administration at bedtime. Unless otherwise specified herein, the term “bedtime” has the normal meaning of a time when a person retires for the primary sleep period during a twenty-four hour period of time. The administration shortly before bedtime means that the FXR agonist as herein defined, is administered within about 1-2 hours prior to a person's normal rest or sleep (typically 4 to 10-hours) period.

### Diseases

As hereinabove defined, the fibrotic or cirrhotic disease or disorder can be a liver disease or disorder, e.g. as defined herein, or renal fibrosis.

As hereinabove defined, the liver diseases or disorders can be cholestasis, intrahepatic cholestasis, estrogen-induced cholestasis, drug-induced cholestasis, cholestasis of pregnancy, parenteral nutrition-associated cholestasis, primary biliary cirrhosis (PBC), primary sclerosing cholangitis (PSC), progressive familiar cholestasis (PFIC), non-alcoholic fatty liver disease (NAFLD), non-alcoholic steatohepatitis (NASH), drug-induced bile duct injury, gallstones, liver cirrhosis, alcohol-induced cirrhosis, cystic fibrosis-associated liver disease (CFLD), bile duct obstruction, cholelithiasis, liver fibrosis, renal fibrosis, dyslipidemia, atherosclerosis, diabetes, diabetic nephropathy, colitis, newborn jaundice, prevention of kernicterus, veno-occlusive disease, portal hypertension, metabolic syndrome, hypercholesterolemia, intestinal bacterial overgrowth, erectile dysfunction, progressive fibrosis of the liver caused by any of the diseases above or by infectious hepatitis. The liver diseases or disorders can also refer to liver transplantation.

As hereinabove defined, the intestinal disease can be idiopathic inflammatory bowel disease, e.g. Crohn's disease or ulcerative colitis.

In one embodiment of the invention, the pharmaceutical compositions (as herein defined) are for the treatment or prevention of a fibrotic disease or disorder, e.g. a liver disease or disorder, e.g. a chronic liver disease, e.g. a liver disease or disorder selected from the group consisting of PBC, NAFLD, NASH, drug-induced bile duct injury, gallstones, liver cirrhosis, alcohol-induced cirrhosis, cystic fibrosis-associated liver disease (CFLD), bile duct obstruction, cholelithiasis, liver fibrosis. In one embodiment of the invention, the pharmaceutical combination (as herein defined) is for the treatment or prevention of fibrosis, e.g. renal fibrosis or liver fibrosis.

According to one embodiment of the invention, the liver diseases or disorders refer to NAFLD, e.g. any stages of NAFLD, e.g. any of steatosis, NASH, fibrosis and cirrhosis.

In one embodiment of the invention, there is provided a FXR agonist of the invention, as herein defined in above listed embodiments for the improvement of liver fibrosis without worsening of steatohepatitis.

In another embodiment of the invention, there is provided a FXR agonist of the invention, as herein defined in above listed embodiments, for obtaining a complete resolution of steatohepatitis without worsening, e.g. improving, of liver fibrosis.

In another embodiment of the invention, there is provided a FXR agonist of the invention, as herein defined in above listed embodiments, for preventing or treating steatohepatitis and liver fibrosis.

In yet another embodiment of the invention, there is provided a FXR agonist of the invention, as herein defined in above listed embodiments for reducing at least one of the features of the NAS score, i.e. one of hepatosteatosis, hepatic inflammation and hepatocellular ballooning; e.g. at least two features of the NAS score, e.g. hepatosteatosis and hepatic inflammation, or hepatosteatosis and hepatocellular ballooning, or hepatocellular ballooning and hepatic inflammation.

In a further embodiment of the invention, there is provided a FXR agonist as herein defined in above listed embodiments, for reducing at least one or two features of the NAS score and liver fibrosis, e.g. for reducing hepatic inflammation and liver fibrosis, or hepatosteatosis and liver fibrosis or hepatocellular ballooning and liver fibrosis.

In yet a further embodiment of the invention there is provided a FXR agonist as herein defined, for treating or preventing, stage 3 fibrosis to stage 1 fibrosis, e.g. stage 3 and/or stage 2 and/or stage 1 fibrosis.

In yet a further embodiment of the invention there is provided a FXR agonist as herein defined, in above listed embodiments for treating or preventing an intestinal disease, e.g. idiopathic inflammatory bowel disease, e.g. Crohn's disease and ulcerative colitis.

### Subjects

According to the invention, the subjects receiving the FXR agonist of the invention can be affected or at risk of a fibrotic disease or disorder, e.g. a liver disease or disorder, e.g. as hereinabove defined.

In some embodiments of the invention, the subject is obese or overweight.

In other embodiments of the invention, the subject may be a diabetic subject, e.g. may have type 2 diabetes. The subject may have high blood pressure and/or high blood cholesterol level.

### Dosing Regimens

Depending on the compound used, the targeted disease or disorder and the stage of such disease or disorder, the dosing regimen, i.e. administered doses and/or frequency may vary. The dosing frequency will depend on; inter alia, the phase of the treatment regimen.

According to the invention, tropifexor (as hereinabove defined), is administered at a dose of about 90 μg to about 250 μg, e.g. about 140 μg to about 200 μg, e.g. about 140 μg. Such doses may be for oral administration. Preferably, tropifexor (as hereinabove defined), is administered at a dose of about 90 μg, or about 140 μg.

In some aspects, tropifexor (as hereinabove defined), is administered at a dose of about 90 μg, about 100 μg, about 110 μg, about 120 μg, about 140 μg, or about 200 μg. Such doses are particularly adapted for oral administration of tropifexor.

In some embodiments, tropifexor, as herein defined, is administered at a dose of about 120 μg delivered orally, of about 140 μg delivered orally or of about 200 μg delivered orally.

In some embodiments, tropifexor as herein defined, is to be administered at a daily dose of about 90 μg.

In some embodiments, tropifexor as herein defined, is to be administered at a daily dose of about 120 μg.

In some embodiments, tropifexor as herein defined, is to be administered at a daily dose of about 140 μg.

In some embodiments, tropifexor as herein defined, is to be administered at a daily dose of about 200 μg.

In some embodiments, tropifexor as herein defined, is to be administered at a daily dose of about 250 μg.

Obeticholic acid is to be administered at a daily dose of about 5 mg, of about 10 mg, of about 15 mg, of about 20 mg, of about 25 mg, of about 30 mg, of about 40 mg or of about 50 mg. In some embodiments, obeticholic acid as herein defined, is to be administered at a daily dose of about 25 mg.

### EXAMPLES

### Example 1: A 2 Week Study in Cynomolgus Monkey Treated with a FXR Agonist

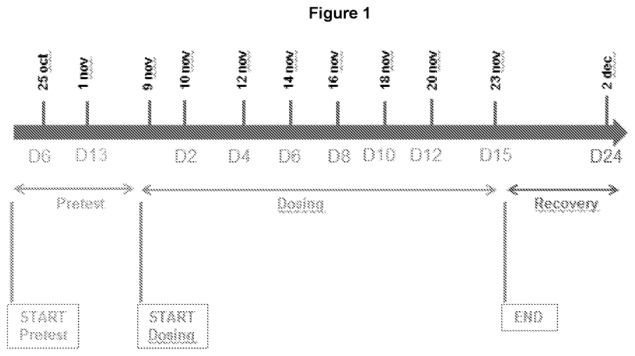

The rate of total bile acid production and the major subsets of the different bile acids have been measured in a 2 week study in Cynomolgus monkey treated with a FXR agonist (LJP305), as shown in FIG. 1 and described in Table 1.

TABLE 1
Study design.

| | | | |
|---|---|---|---|
| | | | Dose |
| | No. of Animals | Dose Level | Concentration |
| Group | Male | (mg/kg/day) | (mg/mL) |

PhaseI

| | | | |
|---|---|---|---|
| 1 LJP305-NX-11 (Low) | 4 | 0.1 | 0.02 |
| 2 LJP305-NX-11 (Mid) | 4 | 1 | 0.2 |
| 3 LJP305-NX-11 (High) | 4 | 3 | 0.6 |

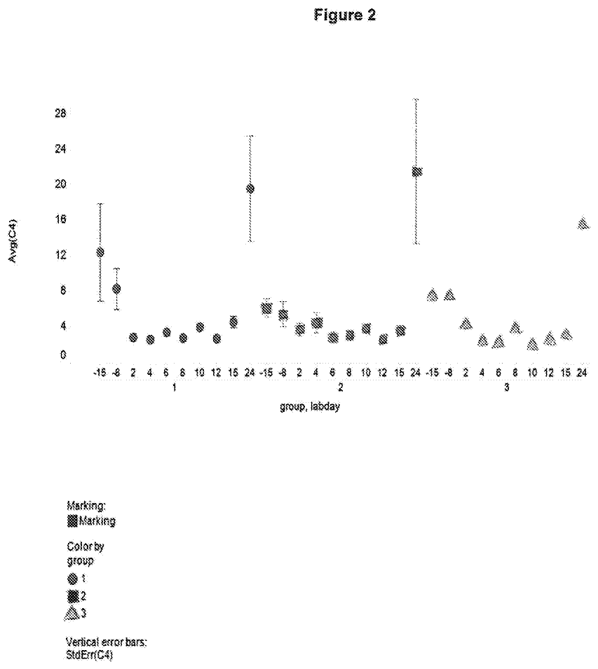

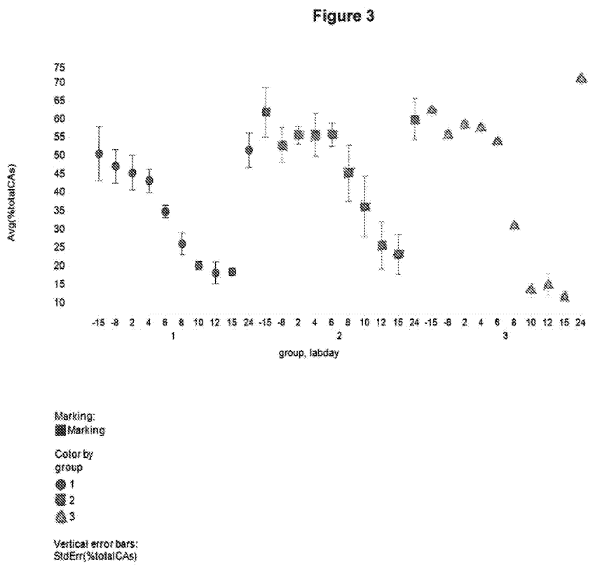

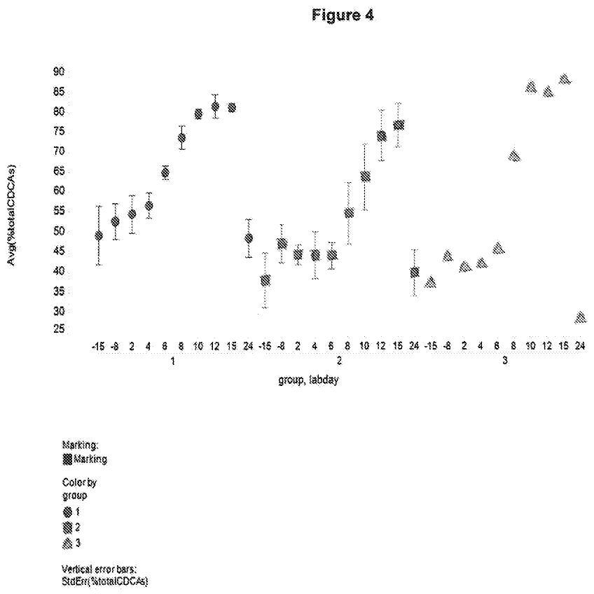

Although total bile acids were decreased (FIG. 2), the ratio of CA to CDCA bile acid was altered overtime with a severe decrease in CA (FIG. 3) but a concomitant increase in CDCA bile acids (FIG. 4).

The most effective method to avoid such an inhibition of Cyp7A1 and consequent activation of the alternate pathway would be to administer an FXR agonist when the enzymatic activity of Cyp7A1 is at the lowest in order to minimize the effect of an FXR-mediated inhibition of the Cyp1A1. As the activity of this enzyme is at the lowest in human during the night, administration of the FXR agonist in the evening (from about 6 pm to about 12 pm, e.g. from about 8 pm to about 11 pm, preferably around 9 pm) should coincide with the time the body naturally decreases the enzyme production/activity and consequently should minimize the impact of such inhibition hence reducing the chance of stimulating the alternate pathway with the resulting production of prurigenic bile acid (CDCA).

### Example 2: In Vitro Human Hepatocytes Treated with FXR Agonists

FXR agonist treatments have been associated, in human, with lipid abnormalities, including increases in peripheral LDL. Increased cholesterol in hepatocytes is associated with a counter mechanism of decrease LDL receptor on the surface of the cells. Such a decrease in the LDL receptor on the surface of the hepatocytes will ultimately results in increases in circulating LDL; the phenotype observed in the clinics.

FIG. 5 shows that in vitro, using in vitro human hepatocytes, the FXR agonists, such as obeticholic acid (OCA) and cilofexor (GS-9674), reduce the LDL uptake by hepatocytes in a dose dependent manner. Those data indicates that blocking of the Cyp7A1 and the bile acid pathway leads to the peripheral increase in LDL. To mitigate the increase in peripheric LDL, we hypothesize that treating the subjects in the evening (from about 6 pm to about 12 pm, e.g. from about 8 pm to about 11 pm, preferably around 9 pm) would be reduce the impact of the drug on LDL. At such time of the day, the level of CYP7A1 are the lowest hence the FRX agonist would have little to no substrate to inhibit hence the inhibition of cholesterol excretion would be at its minimal. In addition, during night time, the hepatocytes rely less on cholesterol coming from the food intake (LDL and others) since the body is then fasting but more on the intrahepatic production of cholesterol via HMGCOa reductase; the activity of this enzyme is the highest during the night. Indeed, in human, whilst the Cyp7A1 activity peak at 1 and 9 pm, intracellular cholesterol levels in hepatocytes are the highest during the night (between midnight and 4 AM).

For high efficacy and/or good safety (e.g. a low risk of itch and/or lipid abnormalities), administration of FXR agonists in the evening is suggested.

### Example 3 Clinical Study for Efficacy, Safety, and Tolerability in Subjects with NASH and Fibrosis (Stage 2 or 3) as per NASH CRN Histological Score

PRIMARY OBJECTIVE: To demonstrate the efficacy of tropifexor as assessed by histologic improvement after 48 weeks of treatment in subjects with NASH and stage 2 or 3 fibrosis.

SECONDARY OBJECTIVES:
Improvement in fibrosis by at least one stage with no worsening of NASH after 48 weeks of treatment
Resolution of NASH with no worsening of fibrosis after 48 weeks of treatment
Improvement in fibrosis by at least one stage
Improvement in fibrosis by at least two stages with no worsening of NASH after 48 weeks of treatment
Reduction in body weight from baseline after 48 weeks of treatment
Change in liver fat content after 48 weeks of treatment
To determine the relationship of investigational treatment and markers of hepatic inflammation in NASH (ALT and AST)
To determine the relationship of investigational treatment and GGT, a marker of cholestasis

The study consists of 1) a screening period, 2) a treatment period starting from randomization on Day 1 and running to Week 48, and 3) a follow up period of 4 weeks after the last dose of study treatment. The screening period starts from the time of the signing of informed consent and continues for up to 8 weeks when all inclusion/exclusion criteria have been evaluated and all baseline assessments have been performed. The study duration from first dose of study medication is 52 weeks. The total duration of participation may be up to 60 weeks.

Subjects eligible for inclusion in this study must meet all of the following criteria:
Written informed consent must be obtained before any assessment is performed.
Male and female subjects 18 years or older (at the time of the screening visit)
Presence of NASH as demonstrated by the following during the screening period: NASH with fibrosis stage 2 or 3 confirmed by central reader's evaluation using NAFLD Activity Score (NAS) and NASH CRN criteria, of liver biopsy obtained no more than 6 months before randomization.
Able to communicate well with the investigator, to understand and comply with the requirements of the study

The planned duration of treatment is 48 weeks. Subjects may be discontinued from treatment earlier due to unacceptable tolerability, disease progression and/or at the discretion of the investigator or the subject.

Subjects (n=70) are assigned at baseline visit to tropifexor monotherapy Arm: tropifexor 140 μg, once daily. Subjects should take the medication in the evening following a meal and at about the same time each day, except at baseline and week 4 where the dose will be taken in the morning at the clinic instead of evening dose.

The efficacy assessments should be completed in the following recommended order:
MRI.
Liver function test: ALT, AST, GGT, total alkaline phosphatase (and isoenzymes if total alkaline phosphatase is>ULN, and 5′nucleotidase if either GGT or total alkaline phosphatase is>ULN during study participation), total bilirubin, and albumin will be assessed.
Protein measurements using SOMAscan.
Markers of liver fibrosis: originally called Fibrotest®/Fibrosure®. The following will be assessed: a2-macroglobulin, apolipoprotein A1, total bilirubin, haptoglobin, GGT, and ALT.
NAFLD fibrosis score: The following formula will be utilized for the calculation of the NAFLD fibrosis score: −1.675+0.037×age (years)+0.094×BMI (kg/m2)+1.13×IFG (increased fasted glucose)/diabetes (yes=1, no=0)+0.99×AST/ALT ratio−0.013×platelet (×109/l)−0.66×albumin (g/dl).
Fasting insulin and glucose: Blood samples will be collected for fasting insulin and glucose assessment.
Liver biopsy: Subjects must have histologic evidence of NASH and liver fibrosis stage 2 or 3 (NASH clinical research network (CRN) staging criteria) demonstrated on liver biopsy within 6 months prior to randomization.

In addition, a Transient Elastography (FibroScan®) can be done at screening/baseline and at the Week 12, 24 and, 48.

Standard safety parameters and measures are collected including adverse events and serious adverse events according to definitions and process detailed in the protocol.

### Example 4: Role of Tropifexor in the Reductions of Hepatic Fat and Serum Alanine Aminotransferase in Patients with Fibrotic NASH after 12 Weeks of Therapy (FLIGHT-FXR Part C Interim Results)

Parts A and B of study CLJN452A2202 in NASH patients have investigated tropifexor at doses ranging from 10 to 90 μg daily for 12 weeks. Tropifexor exhibited a clear dose response for target engagement (FGF19) and biologic activity (GGT). ALT and hepatic fat fraction were reduced across all tropifexor doses (10, 30, 60 and 90 μg) compared to placebo. The study showed that Tropifexor was generally well tolerated up to 90 μg daily without safety signals. Results from the first two parts (A and B, study CLJN452A2202) demonstrated anti-inflammatory and anti-steatotic efficacy of 60 and 90 μg of tropifexor based on biomarkers, and favorable safety at Week 12.

FLIGHT-FXR (NCT02855164) is a phase 2 randomized, double blind, placebo-controlled, 3-part, adaptive-design study to assess the safety, tolerability, and efficacy of several doses of tropifexor (LJN452) in patients with non-alcoholic steatohepatitis (NASH).

METHODS: In Part C, the effects of higher doses of tropifexor on biomarkers and histology will be evaluated over 48 weeks in patients with biopsy-proven NASH and fibrosis stages 2-3. In all, 152 patients (64% females) were randomized to receive placebo (N=51), tropifexor 140 μg (N=50) or tropifexor 200 μg (N=51) once daily. Prespecified endpoints assessed at week 12 included overall safety and changes in alanine aminotransferase (ALT), hepatic fat fraction (HFF), gamma glutamyl transferase (GGT), and body weight.

RESULTS: Prespecified endpoints were met for tropifexor at a dose of 200 μg. Efficacy results are presented in Table 2.

TABLE 2
Least squares means of absolute changes in ALT, GGT,
and body weight, and relative change in HFF from
baseline to Week 12 estimated in repeated measures
or analysis of covariance models (full analysis set)

| | | Tropifexor | Tropifexor |
|---|---|---|---|
| | Placebo | 140 μg | 200 μg |
| Biomarkers | (N = 51) | (N = 50) | (N = 51) |
| ALT (U/L) | −8.9 (4.19) | −20.1 (4.57) | −23.6 (4.48) |
| | n = 49 | n = 41; P = 0.058 | n = 39; P = 0.013 |
| Relative | −10.26 (4.21) | −16.99 (4.64) | −31.37 (4.30) |
| change | n = 51 | n = 49; P = 0.209 | n = 51; P < 0.001 |
| in HFF* | | | |
| (%) | | | |
| GGT (U/L) | −2.5 (3.55) | −39.2 (3.70) | −40.9 (3.62) |
| | n = 49 | n = 44; P < 0.001 | n = 46; P < 0.001 |
| Body weight | −1.14 (0.36) | −2.46 (0.38) | −3.20 (0.37) |
| (kg) | n = 50 | n = 46; P = 0.010 | n = 46; P < 0.001 |

*Measured as magnetic resonance imaging-proton density fat fraction (MRI-PDFF).
Data are presented as LS mean change (SE) with 2-sided P values reported for statistical significance
ALT, alanine aminotransferase; GGT, gamma glutamyl transferase; HFF, hepatic fat fraction; LS, least square; SE, standard error;

Relative HFF reduction (without imputation for missing values) by ≥30% was achieved in 20%, 32%, and 64% of patients in the placebo, Tropifexor 140 μg, and Tropifexor 200 μg groups, respectively. The frequency of serious adverse events was low and comparable across groups. Among patients with pruritus, >60% in both Tropifexor groups and all in the placebo group experienced events with mild (Grade 1) severity. Treatment discontinuation rates due to pruritus were low (Tropifexor 140 μg: n=1 [2%]; Tropifexor 200 μg: n=3 [6%]; placebo: 0%). A dose-related increase in low density lipoprotein-cholesterol (LDL-C) was seen. None of the lipid changes led to treatment discontinuation or dose reduction.

In this prespecified interim analysis of Part C, higher doses of Tropifexor resulted in robust and dose-dependent decreases in ALT, HFF, and body weight with good safety and tolerability after 12 weeks of treatment. Similar to other FXR agonists, these higher doses were associated with mild pruritus and minor dose-related increase in LDL-C.

### Example 5: A Randomized, Investigator and Subject Blinded, Multi-Center, Parallel Arm Study to Determine the Safety and Tolerability of Tropifexor Administered in the Morning or in the Evening to Subjects with NASH

The objective of this study is to determine the effect of tropifexor dosed AM or PM on fasting circulating LDL-C levels, HDL-C after 2 weeks/4 weeks of treatment.

The study consists of a screening period up to 14 days, baseline period up to 21 days, treatment period of 4 weeks followed by a study completion evaluation approximately 30 days after the end of the treatment period. The study population is comprised of male and female adult overweight or obese subjects with EITHER histologic evidence of NASH on liver biopsy within 2 years prior to screening OR phenotypic diagnosis of NASH based on elevated ALT and BMI, diagnosis of Type 2 diabetes (T2D) or currently taking anti-diabetic medications and liver fat content ≥5% by MRI-PDFF. This study investigates if dosing tropifexor in the evening could have advantages over dosing in the morning both in terms of effect on lipids and on pruritus.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

1-20. (canceled)

21. A method for the treatment or prevention of a condition mediated by Farnesoid X receptor (FXR), in a subject in need thereof, comprising administering once daily to said subject a therapeutically effective amount of a FXR agonist, wherein the FXR agonist is administered in the evening.

22. The method of claim 21, wherein said condition mediated by Farnesoid X receptor (FXR) is a liver disease or an intestinal disease.

23. The method according to claim 21, wherein the FXR agonist is selected from tropifexor, obeticholic acid, nidufexor, cilofexor, TERN-101, EDP-305, PXL007, AGN242266 and M ET409.

24. The method according to claim 21, wherein the FXR agonist is obeticholic acid.

25. The method according to claim 24, wherein obeticholic acid is administered at a daily dose of about 5 mg, of about 10 mg, of about 15 mg, of about 20 mg, of about 25 mg, of about 30 mg, of about 40 mg or of about 50 mg.

26. The method according to claim 21, wherein the FXR agonist is tropifexor.

27. The method according to claim 26, wherein tropifexor is administered at a daily dose of about 90 μg to about 250 μg, or about 140 μg to about 200 μg.

28. The method according to claim 26, wherein tropifexor is administered at a dose of about 90 μg/day, of about 140 μg/day, of about 150 μg/day, of about 160 μg/day, of about 170 μg/day, of about 180 μg/day, of about 190 μg/day, of about 200 μg/day, of about 210 μg/day, of about 220 μg/day, of about 230 μg/day, of about 240 μg/day or of about 250 μg/day.

29. The method according to claim 26, wherein tropifexor is administered at a daily dose of about 140 μg.

30. The method according to claim 21, wherein pruritus associated with administration of the FXR agonist is reduced.

31. The method according to claim 21, wherein lipid abnormality associated with administration of the FXR agonist is reduced.

32. The method according to claim 21, wherein said method comprises resolution of steatohepatitis, improvement in liver fibrosis, or resolution of steatohepatitis and improvement in liver fibrosis.

33. A method for the treatment, stabilization or lessening the severity or progression of a non-alcoholic fatty liver disease (NAFLD) in a subject in need thereof, comprising administering once daily to said subject a therapeutically effective amount of a FXR agonist, wherein the FXR agonist is administered in the evening.

34. The method of claim 33, wherein said non-alcoholic fatty liver disease (NAFLD) is non-alcoholic steatohepatitis (NASH).

35. The method according to claim 33, wherein said method further comprises lack of worsening of the subject's NAFLD as defined by Activity (NAS) score, lack of worsening of the subject's Steatosis, Activity and Fibrosis (SAF) Activity score, reduction of liver fat in said subject, improvement in subject's Steatosis, improvement in subject's ballooning, NAFLD resolution, NAFLD resolution without worsening of fibrosis, reduction of fibrosis without NAFLD worsening, reduction of ALT levels in said subject, reduction of AST levels in said subject, reduction of HbA1c levels in said subject, lack of subject's progression to Cirrhosis, inhibiting progression of Non-Alcoholic Fatty Liver Disease (NAFLD) and/or Non-Alcoholic Steatohepatitis (NASH), or any combination thereof.

36. A method for slowing, arresting, or reducing the development of a chronic liver disease or disorder in a subject in need thereof, comprising administering once daily to said subject a therapeutically effective amount of a FXR agonist, wherein the FXR agonist is administered the evening.

37. The method of claim 36, wherein said chronic liver disease or disorder is non-alcoholic fatty liver disease (NAFLD), liver fibrosis or primary biliary cholangitis (PBC).

38. The method of claim 37, wherein said non-alcoholic fatty liver disease (NAFLD) is non-alcoholic steatohepatitis (NASH).

39. A method for reducing cirrhosis or fibrosis in a subject with non-alcoholic fatty liver disease (NAFLD), comprising administering once daily to said subject a therapeutically effective amount of a FXR agonist, wherein the FXR agonist is administered in the evening.

40. The method of claim 39, wherein said non-alcoholic fatty liver disease (NAFLD) is non-alcoholic steatohepatitis (NASH).
